(19) United States
(12) Patent Application Publication
(10) Pub. No.: US 2006/0099237 A1
(43) Pub. Date: May 11, 2006

(54) Antimicrobial medical articles containing a combination of anti-infective compounds, octoxyglycerin, salicylic acid, and sesquiterpenoids

(76) Inventors: Shanta M. Modak, River Edge, NJ (US); Trupti Gaonkar, New York, NY (US)

Correspondence Address: BAKER & BOTTS, 30 ROCKEFELLER PLAZA, NEW YORK, NY, 10112, US

(21) Appl. No.: 11/250,241

(22) Filed: Oct. 14, 2005

Related U.S. Application Data
(63) Continuation of application No. PCT/US04/11806, filed on Apr. 16, 2004.
Application No. PCT/US04/11806 is a continuation-in-part of application No. 10/414,902, filed on Apr. 16, 2003, now abandoned.

(51) Int. Cl. A61K 33/38; A61F 13/00

(52) U.S. Cl. 424/422; 424/618

(57) ABSTRACT

Medical articles impregnated with antimicrobial compositions containing synergistic combinations of octoxyglycerin and other anti-infective compounds are disclosed. Such medical articles may include urinary catheters, central venous catheters, tracheal catheters, arterial grafts, wound dressings, sutures, or any other medical articles derived from polymeric substrates such as biomedical polyurethane, biomedical polyvinylchloride (PVC), biomedical silicon, biodegradable polymers, polytetrafluoroethylene (PTFE), etc. or from natural products including natural rubber, silk or cotton fiber. Antimicrobial compositions comprising salicylic acid and sesquiterpenoids are also disclosed.

### 1. INTRODUCTION

The present invention provides for medical articles impregnated with antimicrobial compositions comprising synergistic combinations of octoxyglycerin and anti-infective compounds. In particular embodiments, the anti-infective compounds comprise chlorhexidine, triclosan, minocycline, trivalent bismuth salts such as bismuth nitrate, the silver compounds silver carbonate or silver sulfadiazine, or various combination thereof. The antimicrobial composition may further comprise organic acids, such as salicylic acid, and sesquiterpenoid compounds, such as farnesol.

The medical articles of the present invention may include urinary catheters, central venous catheters, tracheal catheters, arterial grafts, wound dressings, sutures, or any other medical articles derived from polymeric substrates such as biomedical polyurethane, biomedical polyvinylchloride (PVC), biomedical silicon, biodegradable polymers, polytetrafluoroethylene (PTFE), etc. or from natural products including natural rubber, silk or cotton fiber.

### 2. BACKGROUND OF THE INVENTION

Whenever a medical article comes in contact with a patient, a risk of infection is created. Thus, a contaminated examination glove, tongue depressor, or stethoscope could transmit infection. The risk of infection dramatically increases for invasive medical articles, such as intravenous catheters, arterial grafts, endotracheal or intracerebral shunts and prosthetic devices, which not only are, themselves, in intimate contact with body tissues and fluids, but also create a portal of entry for pathogens.

A number of methods for reducing the risk of infection have been developed which incorporate anti-infective compounds into medical articles. Ideally, such articles provide effective levels of an anti-infective compound during the entire period that the article is being used. This sustained release may be problematic to achieve, in that a mechanism for dispersing an anti-infective compound over a prolonged period of time may be required,. and the incorporation of sufficient amounts of anti-infective compound may adversely affect the surface characteristics of the article. The difficulties encountered in providing effective anti-microbial protection increase with the development of drug-resistant pathogens.

One potential solution to these problems is the incorporation into the medical article of a combination of anti-infective compounds that requires relatively low concentrations of individual anti-infective compounds which may have differing patterns of bioavailability.

Two well known anti-infective compounds are chlorhexidine and triclosan. The following patents and patent applications relate to the use of anti-microbial compounds in medical articles.

U.S. Pat. No. 4,723,950 by Lee relates to a microbicidal tube which may be incorporated into the outlet tube of a urine drainage bag. The microbicidal tube is manufactured from polymeric materials capable of absorbing and releasing antimicrobial substances in a controllable, sustained, time-release mechanism, activated upon contact with droplets of urine, thereby preventing the retrograde migration of infectious organisms into the drainage bag. The microbicidal tube may be produced by one of three processes: (1) a porous material, such as poly-propylene, is impregnated with at least one microbicidal compound, and then coated with a hydrophilic polymer which swells upon contact with urine, causing the leaching-out of the microbicidal compound; (2) a porous material, such as high density polyethylene, is impregnated with a hydrophilic polymer and at least one microbicidal compound; and (3) a polymer, such as silicone, is compounded and co-extruded with at least one microbicidal compound, and then coated with a hydrophilic polymer. A broad range of microbicidal compounds are disclosed, including chlorhexidine and triclosan, and combinations thereof. The purpose of Lee's device is to allow the leaching out of microbicidal compounds into urine contained in the drainage bag; similar leaching of microbicidal compounds into the bloodstream of a patient may be undesirable.

U.S. Pat. No. 5,091,442 by Milner relates to tubular articles, such as condoms and catheters, which are rendered antimicrobially effective by the incorporation of a non-ionic sparingly soluble antimicrobial compound, such as triclosan. The tubular articles are made of materials which include natural rubber, polyvinyl chloride and polyurethane. Antimicrobial compound may be distributed throughout the article, or in a coating thereon. A condom prepared from natural rubber latex containing 1% by weight of triclosan, then dipped in an aqueous solution of chlorhexidine, is disclosed. U.S. Pat. Nos. 5,180,605 and 5,261,421, both by Milner, relate to similar technology applied to gloves.

U.S. Pat. Nos. 5,033,488 and 5,209,251, both by Curtis et al, relate to dental floss prepared from expanded polytetrafluoroethylene (PTFE) and coated with microcrystalline wax. Antimicrobial compounds such as chlorhexidine or triclosan may be incorporated into the coated floss.

U.S. Pat. No. 5,200,194 by Edgren et al. relates to an oral osmotic device comprising a thin semipermeable membrane wall surrounding a compartment housing a “beneficial agent” (that is at least somewhat soluble in saliva) and a fibrous support material composed of hydrophilic water-insoluble fibers. The patent lists a wide variety of “beneficial agents” which may be incorporated into the oral osmotic device, including chlorhexidihe and triclosan.

International Patent Application No. PCT/GB92/01481, Publication No. WO 93/02717, relates to an adhesive product comprising residues of a co-poly-merizable emulsifier comprising a medicament, which may be povidone iodine, triclosan, or chlorhexidine.

U.S. Pat. Nos. 5,019,096 and 5,616,338, both by Fox, Jr. et aL relate to infection-resistant medical articles comprising a synergistic combination of a silver compound (such as silver sulfadiazine) and chlorhexidine and their methods of manufacture, respectively. U.S. Pat. No. 5,334,588 by Fox, Jr. et al. relates to methods of inhibiting transmission of Hepatitis B virus using compositions comprising silver sulfadiazine and preferably further comprising a biguanide such as chlorhexidine and/or a detergent such as sodium deoxycholate.

U.S. Pat. Nos. 5,567,495, 5,772,640, 6,083,208 and 6,106,505 and U.S. patent publication Ser. Nos. 2001/0010016, 2001/0024661, 2002/0122876 and 2002/0173775, all by Modak et aL, provide inter alia for anti-infective medical devices, either hydrophobic or hydrophilic, impregnated, coated, or impregnated and coated with various combinations of chlorhexidine, a silver salt such as silver sulfadiazine, silver oxide, silver carbonate or silver nitrate among others, a bismuth salt such as bismuth nitrate, bismuth citrate or bismuth salicylate among others, a zinc salt, a cerium salt, triclosan, combinations of chlorhexidine free base and chlorhexidine acetate, benzalkonium chloride, citrate, povidoneiodine, parachlorometaxylene, gramicidin, polymixin, norfloxacin, tobramycin, sulfamylon, polyhexamethylene biguanide, alexidine, iodine, rifampicin, miconazole, bacitracin, and minocycline. None of the foregoing patents or published patent applications disclose, teach or suggest the incorporation of octoxyglycerin into such medical devices.

Octoxyglycerin, sold under the trade name Sensiva® SC50 (Schulke & Mayr), is a glycerol alkyl ether primarily used as an emollient. However, octoxyglycerin does exhibit antimicrobial activity against a variety of gram-positive bacteria associated with perspiration odor, such as Micrococcus luteus, Corynebacterium aquaticum, Corynebacteriumflavescens, Corynebacterium callunae, and Corynebacterium nephredi. Consequently, octoxyglycerin is used in various skin deodorant preparations at concentrations between about 0.2 and 3 percent (Sensiva® product literature, Schulke & Mayr).

For example, U.S. Pat. No. 5,885,562 by Lowry et al., issued Mar. 23, 1999, relates to deodorant compositions comprising an antimicrobial agent, namely polyhexamethylene biguanide (at a concentration of between 0.01 and 0.5 percent), together with a polarity modifier such as Sensiva®SC50, at levels of typically 1-15 percent. Compositions disclosed in U.S. Pat. No. 5,885,562 may further comprise a short chain monohydric alcohol such as ethanol at a level of between 20 and 80 percent. Formulations useful as deodorants, however, would differ from those used as antimicrobial agents when impregnated into medical articles, in that formulation useful in medical articles would optimally exhibit rapid broad spectrum activity against bacteria, fungi, and viruses, not merely gram-positive odor causing bacteria.

U.S. Pat. No. 5,516,510 by Beilfuss et aL, issued May 14, 1996, discloses deodorant compositions which comprise glycerin monoalkyl ethers such as octoxyglycerin (referred to therein as 2-ethyl hexyl glycerin ether, and as being the most preferred among these compounds). The deodorant compositions of U.S. Pat. No. 5,516,510 may be formulated in aqueous and/or alcoholic solutions and may further comprise additional antimicrobial compounds, including triclosan, chiorhexidine salts, alexidine salts, and phenoxyethanol, among others. Specific concentration ranges for triclosan and the biguanides are not provided.

U.S. Pat. No. 5,736,574 by Burnier et aL, issued Apr. 7, 1998, discloses that octoxyglycerin synergizes the antimicrobial activity of certain classes of hydrolipids or lipids, and suggests the use of combination of these antimicrobial hydrolipids or lipid and octoxyglycerin in pharmaceutical and/or cosmetic compositions. However, this patent does not disclose, teach or suggest the synergistic properties of octoxyglycerin when used in combination with other antimicrobial compounds. Moreover, this patent does not disclose, teach or suggest the impregnation of these compounds into medical articles.

Various compounds have also been shown to confer enhanced permeability of antimicrobial compounds (Brehm-Stecher et al. “Sensitization of Staphylococcus aureus and Escherichia coli to Antibiotics by the Sesquiterpenoids Nerolidol, Famesol, Bisabolol, and Apritone,” Antimicrob Agents and Chemotherapy Vol. 47. pp. 3357-3360 October 2003). Examples of such compounds include sesquiterpenoid compounds including, inter alia, nerolidol, farnesol, bisabolol, and apritone. Brehm-Stecher et al. show that low concentrations (0.5 to 2 mM) of nerolidol, farnesol, bisabolol and apritone enhanced the susceptibility of S. aureus to a number of clinically important antibiotics as ciprofloxacin, clindamycin, erythromycin, gentamycin, tetracycline and vancomycin. (Brehm-Stecher et al. “Sensitization of Staphylococcus aureus and Escherichia coli to Antibiotics by the Sesquiterpenoids Nerolidol, Farnesol, Bisabolol, and Apritone,” Antimicrob Agents and Chemotherapy Vol. 47. pp. 3357-3360 October 2003). However, the present inventors have observed that the ability of the sesquiterpenoid, farnesol, has only limited ability to enhance the general action of various antimicrobials. For example, there is no enhancement of antimicrobial activity in topical formulations containing compounds, such as triclosan, chlorhexidine gluconate and zinc containing compounds. See pending U.S. Provisional Patent Application Ser. No. 60/530864, filed Dec. 18, 2003, by Modak et aL, which is incorporated by reference herein in its entirety, and discloses that the effects of the ability of farnesol appears to be specific to certain quaternary compounds, and not to all antimicrobial compounds.

In the present invention, it has been discovered that combinations of octoxyglycerin and anti-infective compounds synergistically increase the anti-infective properties of medical articles impregnated and/or coated with various combinations of octoxyglycerin and anti-infective compounds relative to the anti-infective properties of medical articles impregnated and/or coated with the same anti-infective compounds without octoxyglycerin. Pending U.S. patent application Ser. No. 10/047,631 and International PCT Application PCT/US02/33865, both by Modak et al. and both of which are incorporated by reference herein in their entirety, disclose hydroalcoholic antimicrobial compounds comprising octoxyglycerin, a quaternary. ammonium compound, and one or more other antimicrobial agent selected from the group consisting of a biguanide compound, a phenol compound, triclosan, phenoxyethanol, an iodine compound, miconazole, polymyxin, neomycin, minocycline, metal salts such as silver sulfadiazine, and parachlorometaxylenol.

Salicylic acid, which is traditionally used as an antithrombogenic agent, has been recently shown to affect bacterial infection and virulence. In endocarditis, Staphylococcus aureus causes endovascular infections, damaging endothelial cells of valvular tissue. Salicylic acid appears to mitigate the virulent effects, reducing growth and cellular density of S. aureus-induced infective endocarditis in an animal model (Kupferwasser et al., 1999 “Acetylsalicylic Acid Reduces Vegetation Bacterial Density, Hematogenous Bacterial Dissemination, and Frequency of Embolic Events in Experimental Staphylococcus aureus Endocarditis Through Antiplatelet and Antibacterial Effects,” Circulation, 99:2791-2797; Kupferwasser et al., “Salicylic acid attenuates virulence in endovascular infections by targeting global regulatory pathways in Staphylococcus aureus,” J Clin Invest. 2003; 112(2):222-33). In fact, salicylic acid modulates virulence by suppressing expression of adherence factors (Kupferwasser et al., “Salicylic acid attenuates virulence in endovascular infections by targeting global regulatory pathways in Staphylococcus aureus,” J Clin Invest. 2003;112(2):222-33).

U.S. Pat. No. 6,582,719, International Patent Application No. PCT/US02/03087, and pending U.S. patent application Ser. No. 10/633,204, filed Jul. 30, 2003, all of which are incorporated by reference, disclose antimicrobial compositions comprising antiseptics, such as chlorhexidine, triclosan, and benzalkonium chloride, and antibiotics, such as minocycline, which may be particularly useful against antibiotic-resistant microorganisms. It has been discovered, however, that although certain of the chlorhexidine-containing solutions exhibited a broad spectrum of activity against many organisms, the solutions became unstable, forming precipitates after a few days at room temperature. Having a short shelf life limits the application of these compositions in coating and impregnating medical devices.

Therefore, there is a compelling need to develop antimicrobial compositions that are highly effective and remain stable at room temperature. The application of organic acids, such as salicylic acids, may be useful in the maintenance of stability in chlorhexidine-containing solutions.

### 3. SUMMARY OF THE INVENTION

The present invention provides for medical articles impregnated with antimicrobial compositions comprising synergistic combinations of octoxyglycerin and anti-infective compounds. In particular embodiments, the anti-infective compounds comprise chlorhexidine, triclosan, minocycline, trivalent bismuth salts, such as bismuth nitrate, the silver compounds silver carbonate or silver sulfadiazine, or various combination thereof. The antimicrobial composition may further comprise organic acids, such as salicylic acid, and sesquiterpenoid compounds, such as famesol.

The medical articles of the present invention may include urinary catheters, intravenous catheters including arterial catheters and central venous catheters, tracheal catheters, arterial grafts, wound dressings, sutures, or any other medical articles derived from polymeric substrates such as polyvinylchloride (PVC), silicon, polytetrafluoroethylene (PTFE), etc. or from natural products including silk or cotton fiber.

The invention is based, at least in part, on the observation that medical articles impregnated with solutions comprising octoxyglycerin and various combinations of anti-infective compounds display greater antimicrobial properties and maintain these antimicrobial properties for longer periods of time relative to medical articles impregnated with solutions comprising the same combinations of anti-infective compounds but in the absence of octoxyglycerin.

### 4. BRIEF DESCRIPTION OF THE DRAWINGS

FIG. 1. Schematic diagram of an in vitro urinary tract model.

### 5. DETAILED DESCRIPTION OF THE INVENTION

The present invention relates to medical articles comprising synergistic combinations of octoxyglycerin and other anti-infective compounds. Non-limiting examples of anti-infective compounds that may be incorporated into the instant invention include chlorhexidine, triclosan, minocycline, trivalent bismuth salts such as bismuth nitrate, the silver compounds silver carbonate or silver sulfadiazine, or various combinations thereof. The antimicrobial composition may further comprise organic acids, such as salicylic acid, and sesquiterpenoid compounds, such as famesol.

Chlorhexidine may be provided by way of any form, salt or derivative thereof, including but not limited to chlorhexidine free base (CHX) and chlorhexidine salts such as chlorhexidine diphosphanilate, chlorhexidine digluconate, chlorhexidine diacetate (also known as chlorhexidine acetate or CHA), chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluoro-phosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-α-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynaphthoate, and chlorhexidine embonate and combinations thereof. The term “chlorhexidine”, as used herein, may refer to any of such forms, derivatives, or salts, unless specified otherwise. Chlorhexidine salts may be solubilized using polyethylene glycol or propylene glycol, or other solvents known in the art.

The term triclosan (TC) as used herein refers to the compound known alternatively by the chemical names 2,4,4′-trichloro-2′-hydroxydiphenyl ether or 5-chloro-2-(2,4-dichlorophenoxy)phenol. While the present invention is not bound by any particular theory, triclosan is believed to be a biocidal agent, acting to interfere with fatty acid biosynthesis, the first stage in membrane lipid biogenesis.

Minocycline (MN) is an antibiotic related to tetracycline. It is also known by its chemical name 7-dimethylamino-6-deoxy-6-demethyltetracycline. Organisms currently known to be susceptible to minocycline therapy include a wide range of gram-negative and gram-positive bacteria including, but not limited to agents of rickettsiae (Rocky Mountain spotted fever, typhus fever and the typhus group, Q fever, rickettsialpox, tick fevers); Mycoplasma pneumoniae (PPLO, Eaton agent); agents of psittacosis and ornithosis; agents of lymphogranuloma venereum and granuloma inguinale; the spirochetal agent of relapsing fever (Borrelia recurrentis); the agent of Lyme disease (Borrelia burgdorfeni), the agents of acne (Propionibacterium or Corynebacterium acnes); the microorganisms Haemophilus ducreyi (chancroid), Yersinia pestis and Francisella tularensis, formerly Pasteurella pestis and Pasteurella tularensis, Bartonella bacilliformis, Bacteroides species, Vibrio comma and Vibrio fetus, Brucella species, Escherichia coli, Enterbacter aerogenes (formerly Aerobacter aerogenes), Shigella species, Mima species, Herellea species, Haemophilus influenzae (respiratory infections), Klebsiella species (respiratory and urinary infections), many Streptococcus species including strains of Streptococcus pyogenes and Streptococcus faecalis, Streptococcus pneumoniae, Staphylococcus aureus (skin and soft tissue infections), Neisseria gonorrhoeae, Neisseria meningitidis, Treponema pallidum and Treponema pertenue (syphilis and yaws), Listeria monocytogenes, Clostridium species, Bacillus anthracis, Fusobacterium fusiforme (Vincent's infection), Actinomyces species; and in the treatment of acute intestinal amebiasis and inclusion conjunctivitis. Physician's Desk Reference, 1987, Medical Economics Company, Oradell, N.J. (PDR 43rd Ed.).

Octoxyglycerin is a glycerol alkyl ether that exhibits antimicrobial activity against a variety of gram-positive bacteria associated with perspiration odor, such as Micrococcus luteus, Corynebacterium aquaticum, Corynebacterium flavescens, Corynebacterium callunae, and Corynebacterium nephredi. Consequently, octoxyglycerin is used in various skin deodorant preparations at concentrations between about 0.2 and 3 percent (Sensiva® product literature, Schulke & Mayr).

The term silver compound, as used herein, refers to a compound comprising silver, either in the form of a silver atom or a silver ion unlinked or linked to another molecule via a covalent or noncovalent (e.g. ionic) linkage, including but not limited to covalent compounds such as silver sulfadiazine (“AgSD”) and silver salts such as silver oxide (“Ag₂O”), silver carbonate (“Ag₂CO₃”), silver deoxycholate, silver salicylate, silver iodide, silver nitrate (“AgNO₃”), silver paraaminobenzoate, silver paraaminosalicylate, silver acetylsalicylate, silver ethylenediaminetetraacetic acid (“AgEDTA”), silver picrate, silver protein, silver citrate, silver lactate and silver laurate and combinations thereof.

Trivalent salts of bismuth, including bismuth nitrate (Bi(NO₃)₃; BN), are known to those of ordinary skill in the art to be useful as antiseptic and antimicrobial agents. For example, bismuth compounds are used prophylactically to treat diarrhea and, as an iodoform paraffin paste, to limit infection of surgical wounds. In general, bismuth has antibacterial properties with proven medical usefulness, especially in the treatment of Helicobacter pylori. One of ordinary skill in the art would recognize that BN may be replaced by other forms of bismuth, including but not limited to bismuth citrate and bismuth salicylate, and combinations thereof.

The present invention provides for combinations of octoxyglycerin with anti-infective, antimicrobial and antiseptic compounds disclosed herein. The compositions of the present invention may further comprises additional agents that promote antimicrobial activity.

In an embodiment of the invention, the compositions of the present invention may comprise terpenoid compounds. Terpenoids, a broad class of lipophilic secondary metabolites derived from mevalonate and isopentenyl pyrophosphate, occur widely in nature and have been of historical interest to man primarily for their contribution to the characteristic flavors and aromas of herbs, spices and flowers.

In a further embodiment, the compositions of the present invention comprise sesquiterpenoid compounds, which are 15 carbon containing compounds, formed biosynthetically from three 5-carbon isoprene units. Many sesquiterpenoids of plant origin have found use in perfumery and flavoring applications and are now produced industrially from monoterpenoid feedstock. Apart from their use in perfumery, terpenoids have been associated with a variety of important biological functions as pheromones, insect antifeedants, phytoalexins and others. They have also been useful in enhancing the efficacy of antimicrobials, by enhancing the permeability of antimicrobials into the bacterial cells (Brehm-Stecher et al. “Sensitization of Staphylococcus aureus and Escherichia coli to Antibiotics by the Sesquiterpenoids Nerolidol, Farnesol, Bisabolol, and Apritone,” Antimicrob Agents and Chemotherapy Vol. 47. pp. 3357-3360 October 2003). Examples of sesquiterpenoid compounds include, inter alia, nerolidol, farnesol, bisabolol, and apritone etc.

It has been observed these sesquiterpenoid compounds do not have a non-specific and general ability to enhance bacterial permeability and susceptibility to antimicrobial compounds (See pending U.S. Provisional Patent Application Ser. No. 60/530864). In particular, the addition of farnesol to compositions comprising for example, triclosan, chlorhexidine gluconate or zinc pyrithione, do not increase their individual antimicrobial effects when used in a topical formulation.

In yet another embodiment of the invention, the compositions may comprise organic acids, such as, inter alia, salicylic acid. The addition of salicylic acid may serve to promote stability of the compositions disclosed herein.

Medical articles that may be treated according to the invention are either fabricated from or coated or treated with biomedical polymer and include, but are not limited to, catheters including urinary catheters and vascular catheters (e.g. peripheral and central vascular arterial and venous catheters), wound drainage tubes, arterial grafts, soft tissue patches, gloves, shunts, stents, tracheal catheters, wound dressings, sutures, guide wires and prosthetic devices (e.g. heart valves and LVADs). Vascular catheters that may be prepared according to the present invention include, but are not limited to, single and multiple lumen central venous catheters, peripherally inserted central venous catheters, emergency infusion catheters, percutaneous sheath introducer systems and thermodilution catheters, including the hubs and ports of such vascular catheters. As indicated above, the present invention comprises. use of the disclosed compositions to impregnate the medical articles.

The present invention may be further applied to medical articles that have been prepared according to U.S. Pat. Nos. 5,019,096 and 5,616,338, both by Fox, Jr. et al., U.S. Pat. Nos. 5,567,495, 5,772,640, 6,083,208, and 6,106,505, each by Modak et al., and U.S. Patent Publication Ser. Nos. 2001/0010016, 2001/0024661, and 2002/0173775, each by Modak et aL

As used herein, the presently disclosed compositions may refer to either an impregnating solution or a coating solution, wherein the impregnation solution is the solution utilized to impregnate one or more anti-infective compounds into the matrix of a polymer from which a medical article is manufactured, while a coating solution is a solution used to coat an internal or external surface of the medical article with polyurethane or other compounds that regulate the release of the anti-infective compounds from within the matrix of the medical article.

The present invention provides treatment solutions for the impregnation and/or coating of a polymer, said solutions comprising between about 1.0 percent and about 20.0 percent of one or more anti-infective compound and octoxyglycerin.

In preferred embodiments, the anti-infective compound present in the treatment solution is selected from a group consisting of chlorhexidine, triclosan, an antibiotic, a silver compound, a trivalent bismuth salt, and mixtures thereof.

In preferred embodiments, octoxyglycerin is present in one or more of the treatment solutions at concentrations between about 1.0 percent and about 10.0 percent, more preferably between about 3.0 and about 7.0 percent, and most preferably at about 5.0 percent.

In various alternative non-limiting embodiments, the present invention provides solutions for the impregnation of a polymer comprising octoxyglycerin at a concentration between about 1.0 percent and about 10.0 percent, more preferably between about 3.0 and about 7.0 percent, and most preferably at about 5.0 percent, chlorhexidine at a concentration between about 0.5 percent and about 5.0 percent, and preferably between about 1.0 percent and about 4.0 percent; and optionally triclosan at a concentration between about 0.05 percent and about 2.0 percent, and preferably between about 0.05 and about 1.0 percent, wherein a medical article may be dipped or soaked in the impregnating solution. Percentages recited herein refer to percent by weight, except as indicated otherwise.

In another alternative non-limiting embodiment, the present invention provides solutions for the impregnation of a polymer comprising octoxyglycerin at a concentration between about 1.0 percent and about 10.0 percent, more preferably between about 3.0 and about 7.0 percent, and most preferably at-about 5.0 percent, chlorhexidine free base at a concentration between about 0.5 percent and about 5.0 percent, and preferably between about 1.0 percent and about 4.0 percent; and optionally triclosan at a concentration between about 0.05 percent and about 2.0 percent, and preferably between about 0.05 and about 1.0 percent, wherein a medical article may be dipped or soaked in the impregnating solution.

In another alternative non-limiting embodiment, the present invention provides solutions for the impregnation of a polymer comprising octoxyglycerin at a concentration between about 1.0 percent and about 10.0 percent, more preferably between about 3.0 and about 7.0 percent, and most preferably at about 5.0 percent, a chlorhexidine salt at a concentration between about 0.5 percent and about 5.0 percent, and preferably between about 1.0 percent and about 4.0 percent; and optionally triclosan at a concentration between about 0.05 percent and about 2.0 percent, and preferably between about 0.05 and about 1.0 percent, wherein a medical article may be dipped or soaked in the impregnating solution.

In another alternative non-limiting embodiment, the present invention provides solutions for the impregnation of a polymer comprising octoxyglycerin at a concentration between about 1.0 percent and about 10.0 percent, more preferably between about 3.0 and about 7.0 percent, and most preferably at about 5.0 percent, a mixture of chlorhexidine free base and a chlorhexidine salt at a concentration between about 0.5 percent and about 5.0 percent, and preferably between about 1.0 percent and about 4.0 percent; and optionally triclosan at a concentration between about 0.05 percent and about 2.0 percent, and preferably between about 0.05 and about 1.0 percent, wherein a medical article may be dipped or soaked in the impregnating solution.

In preferred embodiments, the impregnating solution further comprises between about 10 percent to about 50 percent (volume/volume) of methanol and between about 50 percent and about 90 percent (volume/volume) of tetrahydrofuran (THF).

In further preferred embodiments, the impregnating solution comprises octoxyglycerin, chlorhexidine free base, a chlorhexidine salt, or mixtures of chlorhexidine free base and chlorhexidine salt, and one or more of another anti-infective compound, including but not limited to chlorhexidine, chlorhexidine free base, a chlorhexidine salt, triclosan, an antibiotic, a silver compound, a trivalent bismuth salt, and mixtures thereof. For example, in certain preferred embodiments, the impregnating solution comprises octoxyglycerin at a concentration between about 1.0 percent and about 10.0 percent, more preferably between about 3.0 and about 7.0 percent, and most preferably at about 5.0 percent; chlorhexidine free base, a chlorhexidine salt, or a mixture thereof at a concentration between about 0.5 percent and about 5.0 percent, and preferably between about 1.0 percent and about 4.0 percent; optionally triclosan at a concentration between about 0.05 percent and about 2.0 percent, and preferably between about 0.05 and about 1.0 percent; and the antibiotic minocycline (MN), at a concentration between about 0.5 percent and about 2.0 percent and most preferably at a concentration of about 1.0 percent.

In another preferred embodiment, the impregnating solution comprises octoxyglycerin at a concentration between about 1.0 percent and about 10.0 percent, more preferably between about 3.0 and about 7.0 percent, and most preferably at about 5.0 percent; chlorhexidine free base, a chlorhexidine salt, or a mixture thereof at a concentration between about 0.5 percent and about 5.0 percent, and preferably between about 1.0 percent and about 4.0 percent; optionally triclosan at a concentration between about 0.05 percent and about 2.0 percent, and preferably between about 0.05 and about 1.0 percent; and a trivalent bismuth salt such as bismuth nitrate (Bi(NO₃)₃; BN), at concentrations between about 0.5 percent and about 2.0 percent and most preferably at a concentration of about 0.75 percent.

In another preferred embodiment, the impregnating solution comprises octoxyglycerin at a concentration between about 1.0 percent and about 10.0 percent, more preferably between about 3.0 and about 7.0 percent, and most preferably at about 5.0 percent; chlorhexidine free base, a chlorhexidine salt, or a mixture thereof at a concentration between about 0.5 percent and about 5.0 percent, and preferably between about 1.0 percent and about 4.0 percent; optionally triclosan at a concentration between about 0.05 percent and about 2.0 percent, and preferably between about 0.05 and about 1.0 percent; the antibiotic minocycline (MN), at a concentration between about 0.5 percent and about 2.0 percent and most preferably at a concentration of about 1 percent; and a trivalent bismuth salt such as bismuth nitrate (Bi(NO₃)₃; BN), at concentrations between about 0.5 percent and about 2.0 percent and most preferably at a concentration of about 0.75 percent.

In particularly preferred embodiments, the chlorhexidine is present in the impregnating solution as a mixture of chlorhexidine free base (CHX) and chlorhexidine acetate (CHA). For impregnation into latex urinary catheters, the preferred concentration of chlorhexidine is between about 1.0 percent and about 4.0 percent, with the chlorhexidine comprising a mixture of CHX and CHA in ratios between about 1:1 to about 1:2. The most preferred concentrations of CHX and CHA for impregnation into latex urinary catheters are about 1.0 percent and 2.0 percent for CHX and CHA, respectively. For impregnation into silicone urinary catheters, the preferred concentration of chlorhexidine is between about 1.0 percent and about 4.0 percent, with the chlorhexidine comprising a mixture of CHX and CHA in ratios between about 1:1 and about 1:2.2 ratio. The most preferred concentrations of CHX and CHA for impregnation into silicone urinary catheters are about 1.25 percent and 2.75 percent for CHX and CHA, respectively.

In other particularly preferred embodiments, the chlorhexidine is present in the impregnating solution as chlorhexidine acetate (CHA) or chlorhexidine free base (CHX). For impregnation into central venous catheters, the preferred concentration of CHA or CHX is between about 1.0 percent and about 5.0 percent, with a most preferred concentrations of about 3.0 percent.

In another embodiment of the invention, organic acids, such as salicylic acid is present in the impregnating solution. The preferred concentration is 0.5% to 5%, more preferably 1%.

In another embodiment of the invention, sesquiterpenoid compounds such as farnesol is present in the impregnating solution. The preferred concentration is 0.1% to 2%, more preferably 0.5%.

In another preferred embodiment, the impregnating solution comprises octoxyglycerin at a concentration between about 1.0 percent and about 10.0 percent, more preferably between about 3.0 and about 7.0 percent, and most preferably at about 5.0 percent; chlorhexidine free base, a chlorhexidine salt, or a mixture thereof at a concentration between about 0.5 percent and about 5.0 percent, and preferably between about 1.0 percent and about 4.0 percent; salicylic acid at a concentration of between about 0.5 percent and about 5.0 percent, and preferably 1.0 percent; and optionally triclosan at a concentration between about 0.05 percent and about 2.0 percent, and preferably between about 0.05 and about 1.0 percent.

In another preferred embodiment, the impregnating solution comprises farnesol at a concentration between about 0.1 percent and about 2 percent, more preferably 0.5 percent; chlorhexidine free base, a chlorhexidine salt, or a mixture thereof at a concentration between about 0.5 percent and about 5.0 percent, and preferably between about 1.0 percent and about 4.0 percent; and optionally triclosan at a concentration between about 0.05 percent and about 2.0 percent, and preferably between about 0.05 and about 1.0 percent.

In another preferred embodiment, the impregnating solution comprises octoxyglycerin at a concentration between about 1.0 percent and about 10.0 percent, more preferably between about 3.0 and about 7.0 percent, and most preferably at about 5.0 percent; chlorhexidine free base, a chlorhexidine salt, or a mixture thereof at a concentration between about 0.5 percent and about 5.0 percent, and preferably between about 1.0 percent and about 4.0 percent; farnesol at a concentration between about 0.5 percent and about 2 percent, more preferably 0.5 percent; and optionally triclosan at a concentration between about 0.05 percent and about 2.0 percent, and preferably between about 0.05 and about 1.0 percent.

The present invention further provides, in various alternative non-limiting embodiments, solutions for the coating of a polymer, either untreated or previously impregnated with an impregnating solution comprising octoxyglycerin and an anti-infective compound, wherein the medical article may be dipped or coated in the coating solution. Said coating solution comprises a biomedical polyurethane and/or hydrogel, and optionally may further comprise, in certain preferred embodiments, one or more anti-infective compounds and octoxyglycerin.

In one embodiment for use with latex or silicone urinary catheters, the coating solution comprises between about 1.0 percent and about 4.0 percent, and preferably about 1.5 percent, of a hydrogel, including but not limited to Biogel (CR Bard), and between about 1.0 percent and about 4.0 percent, and preferably about 1.5 percent, of a polyurethane, including but not limited to polyurethane 80AE (Thermadics). This coating solution further comprises 30 percent (volume/volume) of methanol and 70% (volume/volume) of tetrahydrofuran (THF).

In a second embodiment for use with polyurethane central venous catheters, the coating solution comprises between about 1.0 percent and about 10.0 percent, and most preferably about 6.0 percent, of a polyurethane, including but not limited to polyurethanes 93A and 60D (Thermadics), wherein said about 6.0 percent polyurethane is comprised of about 4.0 percent polyurethane 93A and 2.0 percent polyurethane 60D. This coating solution further comprises 50 percent (volume/volume) THF and 50 percent (volume/volume) methanol.

In preferred embodiments, the coating solution further comprises a silver compound, including but not limited to silver carbonate (Ag₂CO₃) or silver sulfadiazine (AgSD), at concentrations between about 0.1 percent and about 2.0 percent, and preferably between about 0.5 and about 1.5 percent. In particularly preferred embodiments, the silver compound is present in the coating solution at a concentration of about 0.6 percent if the silver compound is silver carbonate (Ag₂CO₃), or at a concentration of about 1.0 percent if the silver compound is silver sulfadiazine (AgSD).

In embodiments of the instant invention where octoxyglycerin is not present in the impregnating solution, the coating solution further comprises octoxyglycerin at concentrations between about 1.0 percent and about 10.0 percent, and preferably between about 3.0 and about 7.0 percent, and most preferably about 5.0 percent.

The instant invention further provides medical articles treated with the impregnating or coating solutions as set forth above, and articles physically equivalent thereto (i.e. articles prepared by a different method but having essentially the same properties).

As used herein, the terms “treat”, “treated”, etc. refer to coating, impregnating, or coating and impregnating a medical article with polymer/anti-infective agent, and the term treatment solution refers to the impregnating and/or coating solutions used to coat and/or impregnate the medical article.

The term “hydrophilic polymer,” as used herein, refers to polymers which have a water absorption greater than 0.6 percent by weight (and, in preferred embodiments, less than 2 percent by weight, as measured by a 24 hour immersion in distilled water according to ASTM Designation D570-81) including, but not limited to biomedical polyurethanes (e.g. ether-based polyurethanes and ester-based polyurethanes, as set forth in Baker, 1987, in Controlled Release of Biologically Active Agents, John Wiley and Sons, pp. 175-177 and Lelah and Cooper, 1986, Polyurethanes in Medicine, CRC Press, Inc., Fla. pp. 57-67, polyurethanes comprising substantially aliphatic backbones such as Tecoflex™ 93A, polyurethanes comprising substantially aromatic backbones such as Tecothane™, and Pellethane™), polylactic acid, polyglycolic acid, natural rubber latex, and gauze or water-absorbent fabric, including cotton gauze and silk suture material.

In a specific, non-limiting embodiment, the hydrophilic medical article is a latex catheter that has been treated with (i.e. dipped or soaked in) one or more impregnating or coating solutions comprising (i) between about 1.0 percent and about 10.0 percent, preferably between about 2.0 percent and about 6.0 percent, and more preferably about 3.0 percent, of a biomedical polyurethane; (ii) between about 0.5 percent and about 5.0 percent, and preferably between about 1.0 percent and about 4.0 percent, of chlorhexidine; (iii) between about 0.0 percent and about 2.0 percent, and preferably between about 0.05 and about 1.0 percent, of triclosan; (iv) between about 1.0 percent and about 10.0 percent, more preferably between about 3.0 and about 7.0 percent, and most preferably about 5.0 percent, of octoxyglycerin.

In related non-limiting embodiments of the invention, one or more of the impregnating or coating solutions used to treat the latex catheter, said solutions comprising a biomedical polyurethane, the anti-infective compound chlorhexidine with or without triclosan, and octoxyglycerin, may further comprise one or more of the following anti-infective compounds: (i) a silver compound, preferably silver carbonate (AgCO₃) or silver sulfadiazine (AgSD), at concentrations of between about 0.1 percent and about 2.0 percent, and most preferably at concentrations of about 0.6 percent for silver carbonate (Ag₂CO₃) or about 1.0 percent for silver sulfadiazine (AgSD); (ii) the antibiotic minocycline (MN), at concentrations between about 0.5 percent and about 2.0 percent and most preferably at a concentration of about 1.0 percent; or (iii) a trivalent bismuth salt such as bismuth nitrate (Bi(NO₃)₃; BN), at concentrations between about 0.5 percent and about 2.0 percent and most preferably at a concentration of about 0.75 percent.

In another particular non-limiting embodiment, the present invention provides for a hydrophilic polymeric medical article treated by dipping or soaking the article in one or more impregnating or coating solutions of a hydrophobic polymer comprising chlorhexidine, octoxyglycerin, and optionally triclosan, wherein the chlorhexidine, octoxyglycerin, and optionally triclosan are present in amounts such that their combination, in the treated article, has effective antimicrobial activity. The term “hydrophobic polymer,” as used herein, refers to a polymer that has a water absorption of less than 0.6 percent and includes, but is not limited to, silicone polymers such as biomedical silicones (e.g. Silastic Type A) or elastomers (e.g. as set forth in Baker, 1987, in Controlled Release ofBiologically Active Agents, John Wiley and Sons, pp. 156-162), Dacron, polytetrafluoroethylene (PTFE, also “Teflon”), polyvinyl chloride, cellulose acetate, polycarbonate, and copolymers such as silicone-polyurethane copolymers (e.g. PTUE 203 and PTUE 205 polyurethane-silicone interpenetrating polymer).

In a specific, non-limiting embodiment, the medical article is a polyurethane catheter which has been dipped or soaked in a treatment solution comprising (i) between about 1.0 percent and about 10.0 percent, preferably between about 2.0 percent and about 6.0 percent, and more preferably about 3.0 percent, of a biomedical silicone; (ii) between about 0.5 percent and about 5.0 percent, and preferably between about 1.0 percent and about 4.0 percent, of chlorhexidine; (iii) between about 0.0 percent and about 0.5 percent, and preferably between about 0.05 and about 0.2 percent, of triclosan; (iv) between about 1.0 percent and about 10.0 percent, preferably between about 3.0 and about 7.0 percent, and most preferably about 5.0 percent, of octoxyglycerin.

In related non-limiting embodiments of the invention, one or more of the impregnating or coating solutions used to treat the hydrophilic polymeric medical article, said solutions comprising a biomedical silicone, the anti-infective compound chlorhexidine with or without triclosan, and octoxyglycerin, may further comprise may further comprise one or more of the following anti-infective compounds: (i) a silver compound, preferably silver carbonate (AgCO₃) or silver sulfadiazine (AgSD), at concentrations of between about 0.1 percent and about 2.0 percent, and most preferably at concentrations of about 0.6 percent for silver carbonate (Ag₂CO₃) or about 1.0 percent for silver sulfadiazine (AgSD); (ii) the antibiotic minocycline (MN), at concentrations between about 0.5 percent and about 2.0 percent and most preferably at a concentration of about 1.0 percent; or (iii) a trivalent bismuth salt such as bismuth nitrate (Bi(NO₃)₃; BN), at concentrations between about 0.5 percent and about 2.0 percent and most preferably at a concentration of about 0.75 percent.

In another particular non-liniting embodiment, the present invention provides for a hydrophobic polymeric medical article treated by dipping or soaking the article in one or more impregnating or coating solutions of hydrophobic polymer comprising chlorhexidine, triclosan, and octoxyglycerin, wherein the chlorhexidine, triclosan and octoxyglycerin are present in amounts such that their combination, in the treated article, has effective antimicrobial activity.

In a specific, non-limiting embodiment, the medical article is a silicone catheter, a polyvinylchloride catheter, or a teflon graft which has been dipped or soaked in one or more impregnating or.coating solutions comprising (i) between about 1.0 percent and about 10.0 percent, preferably between about 2.0 percent and about 6.0 percent, and more preferably about 3.0 percent, of a biomedical silicone; (ii) between about 0.5 percent and about 5.0 percent, and preferably between about 1.0 percent and about 4.0 percent, of chlorhexidine; (iii) between about 0.0 percent and about 0.5 percent, and preferably between about 0.05 and about 0.2 percent, of triclosan; (iv) between about 1.0 percent and about 10.0 percent, preferably between about 3.0 and about 7.0 percent, and most preferably about 5.0 percent, of octoxyglycerin.

In related non-limiting embodiments of the invention, one or more of the impregnating or coating solutions used to treat the hydrophobic polymeric medical article, said solutions comprising a biomedical silicone, the anti-infective compound chlorhexidine with or without triclosan, and octoxyglycerin, may further comprise one or more of the following anti-infective compounds: (i) a silver compound, preferably silver carbonate (AgCO₃) or silver sulfadiazine (AgSD), at concentrations of between about 0.1 percent and about 2.0 percent, and most preferably at concentrations of about 0.6 percent for silver carbonate (Ag₂CO₃) or about 1.0 percent for silver sulfadiazine (AgSD); (ii) the antibiotic minocycline (MN), at concentrations between about 0.5 percent and about 2.0 percent and most preferably at a concentration of about 1.0 percent; or (iii) a trivalent bismuth salt such as bismuth nitrate (BiNO₃)₃; BN), at concentrations between about 0.5 percent and about 2.0 percent and most preferably at a concentration of about 0.75 percent.

In another particular non-limiting embodiment, the present invention provides for a hydrophobic polymeric medical article treated by dipping or soaking the article in one or more impregnating or coating solutions of hydrophilic polymer comprising chlorhexidine, triclosan, and octoxyglycerin, wherein the chlorhexidine, triclosan and octoxyglycerin are present in amounts such that their combination, in the treated article, has effective antimicrobial activity.

In a specific, non-limiting embodiment, the medical article is a silicone catheter, a polyvinyl chloride catheter, or a Teflon graft which has been dipped or soaked in one or more impregnating or coating solutions comprising (i) between about 1.0 percent and about 10.0 percent, preferably between about 2.0 percent and about 6.0 percent, and more preferably about 3.0 percent, of a biomedical polyurethane; (ii) between about 0.5 percent and about 5.0 percent, and preferably between about 1.0 percent and about 4.0 percent, of chlorhexidine; (iii) between about 0.0 percent and about 0.5 percent, and preferably between about 0.05 and about 0.2 percent, of triclosan; (iv) between about 1.0 percent and about 10.0 percent, more preferably between about 3.0 and about 7.0 percent, and most preferably about 5.0 percent, of octoxyglycerin.

In related non-limiting embodiments of the invention, one or more of the impregnating or coating solutions used to treat the hydrophobic polymeric medical article, said solutions comprising a biomedical polyurethane, the anti-infective compound chlorhexidine with or without triclosan, and octoxyglycerin, may further comprise one or more of the following anti-infective compounds: (i) a silver compound, preferably silver carbonate (AgCO₃) or silver sulfadiazine (AgSD), at concentrations of between about 0.1 percent and about 2.0 percent, and most preferably at concentrations of about 0.6 percent for silver carbonate (Ag₂CO₃) or about 1.0 percent for silver sulfadiazine (AgSD); (ii) the antibiotic minocycline (MN), at concentrations between about 0.5 percent and about 2.0 percent and most preferably at a concentration of about 1.0 percent; or (iii) a trivalent bismuth salt such as bismuth nitrate (Bi(NO₃)₃; BN), at concentrations between about 0.5 percent and about 2.0 percent and most preferably at a concentration of about 0.75 percent.

Medical articles prepared according to the invention may be treated on their external surface, internal surface, or both. For example, and not by way of limitation, where the medical article is a catheter, the internal surface and/or external surface of the catheter may be treated according to the invention. Where it is desired to treat both internal and external surfaces, an open-ended catheter may be placed in a treatment solution such that the treatment solution fills the catheter lumen. If only the external surface is to come in contact with treatment solution, the ends of the catheter may be sealed before it is placed in the treatment solution. If only the internal surface is to come in contact with treatment solution, the solution may be allowed to pass through and fill the lumen but the catheter is not immersed in the treatment solution.

Successful treatment of a medical article with a polymer comprising an anti-infective compound may be problematic, particularly where the medical article has a hydrophobic surface. The adherence of the polymer may depend upon (1) the polymeric matrix in which the anti-infective compound is suspended; (2) compatibility (or lack thereof) between the anti-infective compound-polymeric matrix and the surface of the article; (3) the solvent system; and (4) the thickness of polymer/anti-infective compound desirably applied. Furthermore, the rates of release of various anti-infective compounds from diverse polymers may differ. For example, the rate of release of chlorhexidine from a silicone matrix is faster than the rate of release of silver sulfadiazine from the same matrix.

In order to compensate for this difference, one potential solution would be to increase the amounts of chlorhexidine and silver sulfadiazine in the matrix. Unfortunately, polymers comprising high levels of chlorhexidine and silver sulfadiazine have been found to adhere poorly to silicone catheters. To provide an alternative solution to the problem, two different methods for treating medical articles have been developed: a one-step method, and a two-step method, both of which are set forth below.

According to the one-step method of the invention, a polymeric medical article may be treated with a solution comprising octoxyglycerin and one or more anti-infective compounds, and optionally containing a biomedical polymer, dissolved in one or more solvents, wherein the solvents selected are capable of swelling the polymeric medical article to be treated; such a solution is referred to herein as an “impregnating solution”, and the process by which the article is treated with anti-infective compound is referred to as “impregnation”. Suitable solvents include, but are not limited to, tetrahydrofuran (THF), dichloromethane, carbon tetrachloride, methanol, ethanol, methyl ethyl ketone, heptane, and hexane, and mixtures thereof. The biomedical polymer may be hydrophilic or hydrophobic, and includes the various polymers set forth above.

If a hydrophilic polymeric medical article is to be impregnated with chlorhexidine, triclosan, and octoxyglycerin, the impregnating solution may, in specific non-limiting embodiments, comprise the following (percentages of solvents in this paragraph being volume/volume): (1) 80% ethanol/20% THF; (2) 70% THF/30% methanol; (3) 85% THF/15% methanol; (4) 50% THF/50% methanol; (5) 70% THF/30% methanol containing 2-3% of a biomedical polyurethane; (6) 90% reagent alcohol/10% THF; or (7) 100% reagent alcohol. Preferred soaking times vary between 5 minutes and 120 minutes, or until complete swelling of the polymeric medical article is achieved.

In specific, non-limiting embodiments of the invention, a hydrophilic medical article such as a polyurethane catheter may be impregnated using a solvent mixture of 15% methanol/85% THF and 50% methanol/50% THF and chlorhexidine, triclosan and octoxyglycerin for between 5 minutes and 120 minutes. The article may then be air-dried at room temperature for 24 hrs to 48 hrs.

If a hydrophobic polymeric medical article is to be impregnated with chlorhexidine, triclosan and octoxyglycerin, the impregnating solution may, in specific non-limiting embodiments, comprise the following (percentages of solvents in this paragraph being volume/volume): (1) 10% methanoV90% THF; (2) 10% ethanol/90% THF; (3) 30% methanol/70% THF; (4) 15% methanol/85% THF (5) 30% ethanol/70% THF; (6) 1-10 percent silicone polymer in 10% methanol/90% THF; (7) 1-10 percent silicone polymer in 10% ethanol/90% THF; (8) 1-2 percent polylactic acid in 10% methanol/90% THF; (9) 1-2 percent polylactic acid in 10% ethanol/90% THF; (10) 1-5 percent silicone polymer in 30% methanol/70% THF; (11) 1-5 percent silicone polymer in 30% ethanol/70% THF; (12) 1-2 percent polylactic acid in 30% methanol/70% THF; (13) 1-2 percent polylactic acid in 30% ethanol/70% THF; (14) 1-5 percent silicone polymer in 100% methyl ethyl ketone; and (15) 1-2 percent polyurethane in 30% ethanol/70% THF.

In various specific embodiments, the impregnating solution comprises between about 0.2 percent and about 20 percent of a synergistic mixture of one or more anti-infective compound and octoxyglycerin, and between about 0 percent and about 10 percent of a biomedical polymer.

The medical article, or a portion thereof, may be immersed in the impregnating solution to swell, after which the article may be removed and dried at room temperature until all solvent has evaporated and the article is no longer swollen. During the swelling process, anti-infective compound (and small amounts of polymer when present in the impregnating solution) may be distributed within the polymeric substrate of the article; during drying, the anti-infective agent and biomedical polymer (where present) may migrate somewhat toward the surface of the article. After drying, the article may be rinsed in either water or alcohol and wiped to remove any excess anti-infective compound and/or polymer at the surface. This may leave a sufficient amount of anti-infective compound just below the surface of the article, thereby permitting sustained release of the compound over a prolonged period of time. Anti-infective compounds that may be incorporated by this process include but are not limited to chlorhexidine, triclosan, silver compounds, minocycline or other antibiotics, trivalent bismuth compounds, parachlorometaxylene, benzalkonium chloride or other quaternary ammonium compounds, bacitracin, polymyxin, miconasole and rifampicin, as well as combinations thereof.

In preferred, non-limiting embodiments of the invention, synergistic combinations of chlorhexidine, triclosan and octoxyglycerin may be dissolved in a mixture of methanol and tetrahydrofuran to produce an impregnating solution that may be used to render a silicone catheter anti-infective.

In one specific, non-limiting example, the impregnating solution for use in the one-step method comprises between about 1.0 percent and about 10.0 percent, preferably between about 2.0 percent and about 6.0 percent, and more preferably about 3.0 percent, of a biomedical polyurethane; (ii) between about 0.5 percent and about 5.0 percent, and preferably between about 1.0 percent and about 4.0 percent, of chlorhexidine; (iii) between about 0.05 percent and about 2.0 percent, and preferably between about 0.05 and about 1.0 percent, of triclosan; (iv) between about 1.0 percent and about 10.0 percent, more preferably between about 3.0 and about 7.0 percent, and most preferably about 5.0 percent, of octoxyglycerin.

According to the two-step method of the invention, the one-step method may be used to impregnate a medical article with anti-infective agent, and then the medical article may be dipped into a polymeric solution and dried. This method forms a polymeric coating on the article and further controls the rate of release of anti-infective agent. When the two-step method is practiced, the biomedical polymer may be omitted from the first impregnating step. Optionally, an anti-infective compound may be comprised in the polymeric coating.

In a specific, non-limiting example, a latex urinary catheter may be soaked in a mixture of methanol (15% volume/volume) and tetrahydrofuran (80% volume/volume) containing between about 0.5 percent and about 5.0 percent, and preferably between about 1.0 percent and about 4.0 percent, of chlorhexidine, between about 0.05 percent and about 2.0 percent, and preferably between about 0.05 and about 0.5 percent, of triclosan, and between about 1.0 percent and about 10.0 percent, more preferably between about 3.0 and about 7.0 percent, and most preferably about 5.0 percent, of octoxyglycerin for about 30 minutes to about 60 minutes, removed, rinsed in distilled deionized water, and dried at room temperature.

A coating then may be applied by dipping the impregnated medical article into a coating solution comprised of 30% (volume/volume) methanol/70% (volume/volume) THE containing about 1.5 percent of a hydrogel and about 1.5 percent of a biomedical polyurethane, removed and air-dried at room temperature for 24 hr to 48 hr.

Alternatively, the coating solution may comprise between about 1.0 percent and about 20.0 percent of a medical silicone.

Additional anti-infective compounds may be incorporated into either the impregnating solution or the coating solution.

Anti-infective medical articles prepared by other methods (e.g. extrusion, casting, etc.) but being otherwise substantially the same as articles produced by dipping or soaking are within the scope of the claimed invention.

### 6. WORKING EXAMPLES

### 6.1. Example 1

Synergistic activity of octoxyglycerin in combination with the antimicrobial compounds chlorhexidine, triclosan and a silver salt when impregnated into both the internal and external surfaces of urinary catheters

Impregnation of Urinary Catheters.

There were two steps in the impregnation procedure of urinary catheters. In the first step, both the internal and external surfaces of urinary catheters were impregnated with a solution of the following antimicrobial compounds: chlorhexidine (a mixture of chlorhexidine free base [CHX] and chlorhexidine acetate [CHA]) and triclosan (TC). In the second step, a polymer coating was applied to the internal and external surfaces of the impregnated catheter by dipping the catheters in a solution of 1.5% hydrogel and 1.5% polyurethane 80AE to control the release of the antimicrobial compounds from the catheter matrix. Octoxyglycerin (O) and a silver salt could be impregnated into the catheter through their addition to either the impregnation solution or the polymer coating solution.

The following steps were employed to prepare the catheters: 1) prepare 100 ml of the impregnation solution as follows: a) uniformly suspending the antimicrobial compounds chlorhexidine (1.0% CHX and 2.0% CHA for latex catheters and 1.25% CHX and 2.75% CHA for silicone catheters) and triclosan (0.1% for latex catheters and 0.5% for silicone catheters) in a solution containing 15 ml of methanol and 5 ml of octoxyglycerin by vortexing, b) slowly adding 80 ml of tetrahydrofuran (THF) to the solution, c) stirring continuously until the solution clears; 2) prepare 100 ml of the polymer coating solution as follows: a) dissolving hydrogel (Biogel, CR Bard; 1.5%) and Polyurethane 80AE (Thermadics; 1.5%) in a solution of 70% THF and 30% methanol, and b) further dissolving silver carbonate (AgCO₃; 0.6%) in the solution of 70% THF/30% methanol if the medical article is to contain a silver salt; 3) soak the catheter in the impregnation solution for 30 to 60 minutes, by which time maximum uptake of the anti-infective compounds into the catheter are achieved; 4) remove the impregnated catheter from the impregnation solution; 5) rinse the impregnated catheter with deionized water; 6) allow the rinsed, impregnated catheter to de-swell and dry at room temperature; 7) dip the dry, rinsed, impregnated catheter into the polymer coating solution; 8) immediately remove the catheter from the polymer coating solution; and 9) air dry the catheter for 24 hrs at room temperature.

Eight different groups of catheters were made using the process described immediately above:

Group 1—Latex catheters impregnated with 1.0% CHX, 2.0% CHA and 0.1% TC;

Group 2—Latex catheters impregnated with 1.0% CHX, 2.0% CHA, 0.1% TC and 0.6% AgCO₃;

Group 3—Latex catheters impregnated with 1.0% CHX, 2.0% CHA, 0.1% TC, and 5% O;

Group 4—Latex catheters impregnated with 1.0% CHX, 2.0% CHA, 0.1% TC, 0.6% AgCO₃ and 5% O;

Group 5—Silicone catheters impregnated with 1.25% CHX, 2.75% CHA and 0.5% TC;

Group 6—Silicone catheters impregnated with 1.25% CHX, 2.75% CHA, 0.5% TC and 0.6% AgCO₃;

Group 7—Silicone catheters impregnated with 1.25% CHX, 2.75% CHA, 0.5% TC, and 5% 0;

Group 8—Silicone catheters impregnated with 1.25% CHX, 2.75% CRA, 0.5% TC, 0.6% AgCO₃ and 5% 0.

In Vitro Urinary Tract Model.

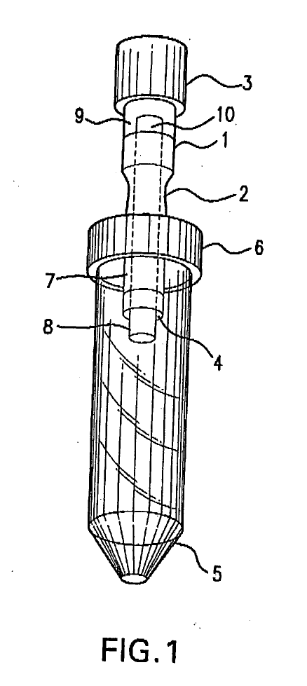

The in vitro urinary tract model is shown in FIG. 1. This model consists of two tubes, an inner cylindrical tube (1), circumferentially crimped at the center (2), with one end capped (3) and one open end (4), inserted into the interior of an outer tube (5) through the capped end (6) of an outer tube (5). The inner cylindrical tube (1) contains within its length a toroidal plug of agar (7) through the center of which runs the test catheter (8). The system is designed to simulate the anatomy of the urinary tract, with the space (9) the inner tube (1) above the agar representing the urinary bladder, the agar (7) surrounding the test catheter (8) simulating the urethra and the lower end of the agar plug (7) representing the meatus of the urinary tract.

To assemble the model, the following steps were performed: 1) the inner tube (1) was capped at one end (3) and a rubber stopper with a hole in the center was inserted into the open end (4) of the inner tube (1); 2) both the inner tube (1) and the outer tube (6) were sterilized with ethylene dioxide; 3) catheter segments (8) of 6 cm in length, with both ends sealed with silicone to prevent intraluminal bacterial contamination were sterilized with ethylene dioxide; 4) the sterilized catheter segment was inserted into the inner tube (1) by aseptically lifting the cap (3) and directing the catheter longitudinally through the tube and exiting through the hole in the rubber stopper at the open end (4); 4) sterile modified trypticase soy agar (TSA) media (1% Bacto agar+4% TSA) was prepared, cooled to 40° C., and poured into the inner tube (1) by aseptically lifting the cap (3) and pouring the agar along the inner surface of the outer tube, leaving the upper 1 cm of the catheter protruding out into the space (10) above the agar tract; 5) following solidification of the agar, the rubber stopper at the open end (4) of the inner tube (1) was removed gently without disturbing the agar column on the top, exposing the lower end of the catheter (8); 6) the inner tube (1) then was inserted into the outer tube (5) through a hole in the capped end (6) of the outer tube (5).

Inoculation and Determination of Bacterial Growth.

The meatus of the in vitro urinary tract model was inoculated daily with 20 μl of a solution containing 10⁷ colony forming units (cfu) per ml of a test organism after-removal of Tube 2. The bladder chamber of the in vitro urinary tract model was filled daily with fresh sterile urine. At the end of each 24 hr period, loop cultures taken daily from upper and lower surfaces of the agar plug, representing the bladder and the meatus, respectively, were cultured on TSA to determine the presence of bacterial growth.

On days in which a positive culture was obtained from the bladder sample, the corresponding catheter segment also was examined for determination of bacterial colonization on the catheter surface. In these studies, the catheter segment (8) was removed from the bladder end of inner tube (1), rinsed with saline, and rolled on the agar surface of a culture plate containing drug neutralizing agar (D/E). The inoculated plate was then incubated for 24 hours at 37° C. to determine semi-quantitatively the presence of bacteria on the surface of the catheter. The results of these studies are shown in Table 1.

TABLE 1
Duration of antimicrobial efficacy (in days) against various
uropathogens in the in vitro urinary tract model

| | Antimicrobial Composition of Impregnating Solution | | | | | | | |
|---|---|---|---|---|---|---|---|---|
| | Latex Catheters | | | | Silicone Catheters | | | |
| Organism | Group 1 | Group 2 | Group 3 | Group 4 | Group 5 | Group 6 | Group 7 | Group 8 |
| Gram-positive | | | | | | | | |
| E. faecalis | 2 | 2 | >29 | >29 | 3 | 4 | >31 | >31 |
| Gram-negative | | | | | | | | |
| K. pneumoniœ | 2 | 2 | >29 | >29 | 5 | 5 | 30 | 30 |
| E. aerogenes | 3 | 3 | >29 | >29 | 2 | 3 | 8 | 8 |

Results.

Positive cultures of both gram-positive and gram-negative bacteria were obtained from both latex and silicone catheters impregnated with the antimicrobial agents chlorhexidine and triclosan within 2-5 days of the introduction of these various uropathogens onto the meatus of the in vitro urinary bladder model. In contrast, the addition of 5% octoxyglycerin to the catheter increased the duration of the anti-microbial effects of chlorhexidine and triclosan to greater than 29 days against both gram-positive and gram-negative bacteria for latex catheters and to greater than 31 days against gram-positive bacteria for silicone catheters. A variable antimicrobial effect was observed against gram-negative bacteria using silicone catheters impregnated with the antimicrobial compounds.

An antimicrobial effect similar to that observed for gram-positive bacteria in silicone catheters also was observed for the gram-negative bacterial strain K pneumonia, in silicone catheters, but a much weaker antimicrobial effect was observed for the gram-negative bacterial strain E. aerogenes. However, it is clear from these studies that urinary latex and silicone catheters prepared in accordance with the instant invention exhibited antimicrobial efficacy against gram-positive and gram-negative bacteria for surprisingly long periods of time. For example, a greater than 29 day duration of antimicrobial efficacy in latex urinary catheters impregnated with chlorhexidine, triclosan and octoxyglycerin (Group 3 in Table 1) was achieved as compared with the antimicrobial efficacy of three days or less observed in urinary latex catheters impregnated with only chlorhexidine and triclosan (Group 1 in Table 1). For each catheter group, samples were tested in duplicate and the mean is reported.

### 6.2. Example 2

Synergistic activity of octoxyglycerin in combination with the antimicrobial compounds chlorhexidine and minocycline when impregnated into the external surface of central venous catheters

Impregnation of Central Venous Catheters.

Polyurethane central venous catheters were prepared according to the following one-step method: 1) Prepare 100 ml of an impregnation solution as follows: a) uniformly suspend various mixtures of the antimicrobial compounds chlorhexidine acetate (CHA; 3.0%) or chlorhexidine free base (CHX; 3%), minocycline (MN; 1.0%), triclosan (TC; 1.0%), or octoxyglycerin (3%) in 100 ml of 50% THF/50% methanol; b) further dissolve into the impregnation solution polyurethane 93A (4%) and polyurethane 60D (2%); 2) dip the catheter into the impregnation solution; 3) immediately remove the catheter from the impregnation solution; and 4) air dry the catheter for 24 hrs at room temperature.

Model for Evaluation of the Antimicrobial Efficacy in Central Venous Catheters.

At the end of the drying period, catheter segments were tested for their efficacy against the adherence of the various organisms indicated in Table 2 as follows: 1) trypticase soy agar(TSA) plates were seeded on their agar surfaces with approximately 3.0×10⁷ cfu of the various organisms indicated in Table 2; 2) three 0.5 cm-long catheter segments from each of the treatment groups indicated below were embedded vertically into the agar plates; 3) the agar plates containing the embedded catheter segments were incubated for 24 hours at 37° C.; and 4) the diameter of the zone of inhibition of bacterial growth surrounding each catheter segment was then measured.

Antimicrobial Efficacy in Central Venous Catheters.

Various different groups of catheters were made using the process described above. These catheters were impregnated with the following combinations of anti-infective compounds:
Group 9—3% CHA, 1% MN;
Group 10—3%CHA, 1% MN,3% O
Group 11—3% CHX, 1% MN;
Group 12—3% CHX, 1% MN, 3% O;
Group 13—3% CHA, 1% MN, 1% TC;
Group 14—3% CHA, 1% MN, 1% TC, 3% O
Group 15—3% O
Group 16—1.5% CHX, 1.5% CHA;
Group 17—1.5% CHX, 1.5% CHA, 3% O.

The impregnated catheters were then examined for antimicrobial efficacy using the model described immediately above. The results of these studies are illustrated in Table 2 below.

TABLE 2
Antimicrobial efficacy of octoxyglycerin when impregnated
into central venous catheters either alone or in combination
with other antimicrobial agents

| | Zone of inhibition (mm) in catheters | | | | | | | | |
|---|---|---|---|---|---|---|---|---|---|
| | of various treatment groups | | | | | | | | |
| Organism | 9 | 10 | 11 | 12 | 13 | 14 | 15 | 16 | 17 |
| P. aeruginosa | 8.0 | 8.5 | 9.0 | 12.0 | 9.0 | 11.0 | 0 | 7.0 | 8.5 |
| C. albicans | — | — | — | — | — | — | 0 | 7.0 | 9.0 |
| E. aerogenes | 9.0 | 11.0 | 8.5 | 11.5 | 9.0 | 11.0 | 0 | — | — |
| K. pneumoniœ | 10.0 | 11.5 | 10.5 | 12.0 | 10.0 | 11.0 | 0 | — | — |
| A. baumani | 18.0 | 19.5 | 18.8 | 20.0 | 18.0 | 18.5 | 0 | — | — |
| E. coli | 10.0 | 12.0 | 10.0 | 12.0 | 11.0 | 12.0 | 0 | — | — |
| E. faecalis | — | 16.0 | — | — | 15.0 | 16.0 | 0 | — | — |
| Vancomycin- | — | 11.0 | — | — | 7.5 | 9.5 | 0 | — | — |
| resistant | | | | | | | | | |
| E. faecalis | | | | | | | | | |

As shown in Table 2, the addition of octoxyglycerin to an impregnation solution containing CHA+MN, CHX+MN, CHA+MN+TC, or CHX+CHA (Groups 10, 12, 15, or 17, respectively, in Table 2) exerted a surprisingly enhanced antimicrobial effect against a broad spectrum of organisms over that observed in catheters treated with CHA+MN, CHX+MN, CHA+MN+TC, or CHX+CHA without octoxyglycerin (Groups 9, 11, 13 or 16, respectively, in Table 2). No inhibition of bacterial growth was observed surrounding catheter segments impregnated with octoxyglycerin alone (Group 15 in Table 2). Thus, the addition of octoxyglycerin potentiates the antimicrobial effects of CHA+MN, CHX+MN, CHA+MN+TC, or CHX+CHA against a wide spectrum of bacterial agents.

### 6.3. Example 3

Synergistic activity of octoxyglycerin in combination with the antimicrobial compounds chlorhexidine and minocycline, either alone or in combination with bismuth nitrate or triclosan, on the adherence of P. aeruginosa in central venous catheters

Impregnation of Central Venous Catheters.

The catheters were impregnated with anti-infective compound according to the one-step method described above in Example 6.2.

Agar Tract Model.

Culture tubes containing 12 ml of agar media (1.0% Bacto agar, obtained from Difco, plus 20% bovine adult serum (Sigma)+0.5% Parmalat milk+0.03% TSB in phosphate-buffered saline) were prepared. Heat-sealed segments of test catheters, 4 cm in length and impregnated with various combinations of antimicrobial agents as described above in Example 6.2, were inserted vertically into the agar so that 3.5 cm of the catheter was embedded into the agar and 0.5 cm of the catheter protruded above the agar surface. Six catheter segments were tested for each of the antimicrobial combinations shown in Table 3.

Immediately before inoculation, an additional 0.5 cm of the catheter segment was exposed from the agar and inoculated with 20 μl of a solution containing 10⁸ cfu of P. aeruginosa or S. aureus. The catheter then was returned to its original position and the tube was incubated for 7 days at 37° C. At the end of the incubation period, each of the catheters was removed from the agar and those from each group were combined and rinsed three times with 30 ml saline and blotted gently with a tissue. A 1 cm portion was excised from each end of the catheter and the remaining pieces were suspended in 4 ml of the drug inactivating media LTSB, sonicated for 20 min and 0.5 ml of the resulting solution was plated on a TSA plate. The TSA plates then were incubated at 37° C. for 24 hours and the number of colonies present at the end of the incubation period were determined.

Determination of Bacterial Adherence.

As shown in Table 3, impregnation of the central venous catheter with octoxyglycerin alone had no positive effect on bacterial adherence. Impregnation of the catheter with 3% CHA and 1% MN produced a 10-fold reduction in bacterial adherence, and bacterial adherence could be reduced another 20-fold by the further addition of 5% octoxyglycerin. Octoxyglycerin also potentiated by approximately 5-fold the already strong (approximately 100-fold) antimicrobial effects of 3% CHA, 1% MN and 0.75% bismuth nitrate (BN), 3% CHA, 1% MN and 1% TC, or 3% CHX, 1% MN and 0.75% BN. Thus, octoxyglycerin synergizes the antimicrobial effects of the anti-infective compounds CHX, CHA, MN, BN, TC in central venous catheters.

TABLE 3
Adherence of P. aeruginosa on central venous catheters
impregnated with various antimicrobial compounds infected
immediately after insertion in an agar tract model

| | Adherence of |
|---|---|
| Catheter treatment | P. aeruginosa (cfu/ml) |
| None (control) | 8.0 × 10⁴ |
| 5% octoxyglycerin | 8.2 × 10⁴ |
| 3% CHA + 1% MN | 8.0 × 10³ |
| 3% CHA + 1% MN + 5% O | 3.3 × 10² |
| 3% CHA + 1% MN + 0.75% BN | 8.2 × 10² |
| 3% CHA + 1% MN + 0.75% BN + 5% O | 1.6 × 10² |
| 3% CHA + 1% MN + 1% TC | 7.3 × 10² |
| 3% CHA + 1% MN + 1% TC + 5% O | 1.6 × 10² |
| 3% CHA + 1% MN + 1% TC + 1% O | 4.5 × 10² |
| 3% CHX + 1% MN + 0.75% BN | 6.0 × 10² |
| 3% CHX + 1% MN + 0.75% BN + 5% O | 8.0 × 10¹ |

### 6.4. Example 4

Duration of the synergistic activity of octoxyglycerin in combination with the antimicrobial compounds chlorhexidine (CHX and CHA) and minocycline on the adherence of S. aureus in central venous catheters

In these studies, the catheters were impregnated and inserted into the agar as described immediately above in Example 3. However, the catheters were implanted for 57 days and transferred to fresh media one day prior to inoculation on the 58^{th} day with the bacterial culture solution, rather than immediately after insertion as described above. After inoculation, the catheters were returned to their original positions and incubated for 7 days at 37° C. At the end of the incubation period, each of the catheters was removed from the agar and processed as described above for Example 3.

The results of these studies, shown in Table 4, demonstrate that the synergistic effects of octoxyglycerin on the antimicrobial effects of chlorhexidine and minocycline or chlorhexidine and silver sulfadiazine persist even after 57 days of implantation, indicating that the benefits of the addition of octoxyglycerin to polymeric medical devices containing these other antimicrobials are long-lasting.

TABLE 4
Adherence of S. aureus on central venous catheters impregnated
with various antimicrobial compounds infected 58 days
after insertion in an agar tract model

| | Adherence of |
|---|---|
| Catheter treatment | S. aureus (cfu/ml) |
| None (control) | 8.0 × 10⁴ |
| 3% O | 8.2 × 10⁴ |
| 1.25% CHX + 1.25% CHA + 1% MN | 8.0 × 10³ |
| 1.25% CHX + 1.25% CHA + 1% MN + 3% O | 3.3 × 10² |
| 3% CHA + 0.75% AgSD | 5.5 × 10⁴ |
| 3% CHA + 0.75% AgSD + 3% O | 1.0 × 10⁴ |

### 6.5. Example 5

Efficacy of octoglycerin in combination with antimicrobial compounds, chlorhexidine free base, chlorhexidine salt, triclosan and salicylic acid on bacterial growth.

Impregnation of Central Venous Catheters. The catheters were impregnated with the compounds described below according to the method described in Example 6.2.

Urinary Catheters.

Uncoated latex Foley catheters (16 Fr) were impregnated with antimicrobial compositions listed below and as discussed at page 35, para. 94, except that the catheters were dipped in coating solution containing Polyurethane 93A and Polyurethane ATAE.

1. 1% CHX+2% CHA+0.1% TC+1% SA dissolved in 85% THF and 15% Methanol.

2. 1% CHX+2% CHA+0.1% TC+1% SA+5% Octoxyglycerin dissolved in 80% THF and 15% Methanol.

Three 0.5 cm segments of each type of test catheter or endotracheal tubes (ET) were embedded vertically in modified trypticase soy agar (TSA) media seeded on the surface with 0.3 ml of 10⁸ colony forming units (CFU) per ml of the test organism and the plates were incubated at 37° C. for 24 hours. Diameter of zones of 5 inhibition of bacterial growth around the catheter or ET segments was measured on after one, two and three days of incubation.

TABLE 5
Zone of inhibition data from coated urinary catheter tubes

| | Zone of Inhibition (mm) | | | | | |
|---|---|---|---|---|---|---|
| | S. aureus | | | E. coli | | |
| Group | Day 1 | Day 2 | Day 3 | Day 1 | Day 2 | Day 3 |
| 1 | 16.3 | 15.8 | 15.3 | 15.0 | 12.0 | 11.0 |
| 2 | 20.0 | 20.0 | 19.3 | 14.8 | 14.3 | 13.0 |

Silicone Foley catheters were also impregnated with antimicrobial compositions comprising CHX (0.5-2%), TC (0.1-1%), SA (0.5-2%), and octoxyglycerin (1-5%) as described at page 35, para. 94, except that the polymer coating solution is medical adhesive silicon.

Endotracheal Tubes (ET)

Uncoated endotracheal tubes (Hi-Lo 7.0 mm) were coated with following combinations of antimicrobials.

3. 0.5% CHX+0.5% CHA+1% SA dissolved in 70% THF and 30% Methanol and containing Polyurethane 93A and Polyurethane 6TD.

4. 0.5% CHX+0.5% CHA+1% SA+5% Octoxyglycerin dissolved in 65% THF and 30% Methanol and containing Polyurethane 93A and Polyurethane 6TD.

TABLE 6
Zone of inhibition data from coated endotracheal tubes

| | Zone of Inhibition (mm) | | |
|---|---|---|---|
| Group | E. aerogenes | A. baumanni | S. aureus |
| 3 | 18 | 21.5 | 20.8 |
| 4 | 19.8 | 22 | 24.3 |

Tables 5 and 6 shows that the addition of octoxyglycerin to antirnicrobials in the presence of salicylic acid also produced enhanced antimicrobial activity against various test pathogens in urinary catheters and endotracheal tubes.

### 6.6. Example 6

Duration of antimicrobial efficacy (in days) of octoglycerin in combination with antimicrobial compounds, chlorhexidine free base, chlorhexidine salt, triclosan and salicylic acid against various bacterial pathogens in the in vitro urinary tract model.

In Vitro Urinary Tract Model.

Experiments performed to evaluate duration of the efficacy of the in vitro urinary tract model was applied as described in Example 1 above.

The addition of 5% octoxyglycerin to a composition comprising chlorhexidine free base, chlorhexidine salt, triclosan and salicylic acid, used to coat the catheter increased the duration of the anti-microbial effects of chlorhexidine free base, chlorhexidine salt, triclosan and salicylic acid from 1 to 5 days against P. mirabilis and from 1 to 2 days against P. aeruginosa. Therefore, octoxyglycerin in combination with other antimicrobials exhibited longer efficacy in the in vitro urinary tract agar model

TABLE 7
Duration of antimicrobial efficacy (days)

| Components | P. mirabilis | P. aeruginosa |
|---|---|---|
| 1% CHX + 2% CHA + 0.1% TC + | 1 | 1 |
| 1% SA in 85% THF and 15% Methanol. | | |
| 1% CHX + 2% CHA + 0.1% TC + | 2 | 5 |
| 1% SA + 5% Octoxyglycerin in 80% | | |
| THF and 15% Methanol. | | |
| Control* | 1 | 1 |

*uncoated catheter without antimicrobials

### 6.7. Example 7

Antimicrobial efficacy of octoglycerin in combination with antimicrobial compounds, chlorhexidine free base, chlorhexidine salt, and salicylic acid against various bacterial pathogens in the in vitro airway model.

In Vitro Airway Model

The antimicrobial effect of compositions comprising octoglycerin in combination with antimicrobial compounds, chlorhexidine free base, chlorhexidine salt, triclosan and salicylic acid were evaluated for bacterial colonization on endotracheal (ET) segments in an in vitro airway model as described below.

Method

An airway model comprises a 50 ml sterile tube containing 30 ml of a specially constituted sterile medium (1% Difco agar, 0.03% Trypticase soy agar, 5% Bovine adult serum, 0.5% whole milk UHT (parmalat) in PBS, which was developed to simulate the endotracheal lumen. The agar column is inoculated at the top with 100 μl of the clinical isolate of E. aerogenes (10⁸ cfu/ml). The ET segment is then pushed through the agar column from the top leaving the upper 0.5 cm of the segment protruding out of the agar tract. The proximal end of the ET segment may be thought of as mouth and the distal end inserted into the media as the tracheal portion of airway model. The tubes are incubated at 37° C. for five days and the bacterial colonization on the surface of ET was determined.

After 5 days of incubation, swab cultures were taken from the top (proximal) end of the agar columns holding the tubes and were cultured on TSA. Each ET segment was then removed, gently blotted dry with the tissue paper to remove loosely adhering bacteria as well as the excess medium or any fluid from the surface of the tubes and bacterial colonization was determined semi-quantitatively. The tubes were subcultured on drug inactivating Dey Engley agar (DE agar) by rolling them on the plate. After removing the ET segments from the model, the distal ends of the agar column were swabbed and subcultured on TSA. The plates were incubated at 37° C. for 24 h and colony counts (cfil/tube) were determined. For swab subcultures an approximate estimation of colony counts per plate was recorded.

Results are shown in Tables 8 and 9 below. The combination of chlorhexidine free base, chlorhexidine diacetate, salicylic acid and octoxyglycerin was more effective at preventing bacterial colonization on ET segments than chlorhexidine free base, chlorhexidine diacetate, salicylic acid, without octoxyglycerin.

TABLE 8
Bacterial colonization of ET segments

| | Bacterial colonization |
|---|---|
| Composition | of ET segments |
| 0.5% CHX + 0.5% CHA + 1% SA | 1.7 × 10³ |
| 0.5% CHX + 0.5% CHA + 1% S A + | 2.9 × 10² |
| 5% Octoxyglycerin | |
| Control* | ˜10⁶ |

*Uncoated ET without antimicrobial.

TABLE 9
Growth of cultures swabbed from traches

Subculture from trachea

| Composition | Proximal site | Distal site |
|---|---|---|
| 0.5% CHX + 0.5% CHA + 1% SA | ˜10⁶ | ˜10⁵ |
| 0.5% CHX + 0.5% CHA + 1% SA + | 1 × 10³ | 1.5 × 10² |
| 5% Octoxyglycerin | | |
| Control* | ˜10⁶ | ˜10⁶ |

*Uncoated ET without antimicrobial.

### 6.8. Example 8

Efficacy of farnesol in combination with antimicrobial compounds chlorhexidine free base, chlorhexidine diacetate, and triclosan on bacterial growth.

Impregnation of Central Venous Catheters.

The catheters were impregnated with the compounds described below according to the method described in Example 6.2.

Uncoated latex Foley catheters (16Fr) and endotracheal (ET) segments were impregnated with antimicrobial combinations listed below.

5.1% CHX+2% CHA+0.1% TC dissolved in 85% THF and 15 % Methanol and containing Polyurethane 93A and Polyurethane 6TD.

6. 1% CHX+2% CHA+0.1% TC+0.5% Farnesol dissolved in 84.5% THF and 15% Methanol and containing Polyurethane 93A and Polyurethane 6TD.

7. 0.5% CHX+0.5% CHA dissolved in 70% THF and 30% Methanol and containing Polyurethane 93A and Polyurethane 6TD.

8. 0.5% CHX+0.5% CHA+0.5% Farnesol dissolved in 69.5% THF and 30% Methanol and containing Polyurethane 93A and Polyurethane 6TD.

Tables 10 and 11 show the result of inhibition studies on urinary catheter and ET tubes. The addition of farnesol to compositions comprising of chlorhexidine free base, and chlorhexidine diacetate, show that it is more effective at inhibiting bacterial growth than chlorhexidine free base and chlorhexidine diacetate, without farnesol. Similar results are observed when the compositions further comprise triclosan. Thus, farnesol appears to enhance the antimicrobial activity of chlorhexidine free base, chlorhexidine diacetate and triclosan.

TABLE 10
Zone of inhibition data from coated urinary catheter tubes

| | Zone of Inhibition (mm) | |
|---|---|---|
| Compositions | P. mirabilis | P. aeruginosa |
| 1% CHX + 2% CHA + 0.1% TC (5) | 9.8 | 10.3 |
| 1% CHX + 2% CHA + 0.1% TC + | 11.5 | 11.0 |
| 0.5% Farnesol (6) | | |

TABLE 11
Zone of inhibition data from coated endotracheal tubes

| | Zone of Inhibition (mm) |
|---|---|
| Composition | against E. aerogenes |
| 0.5% CHX + 0.5% CHA (7) | 8.0 |
| 0.5% CHX + 0.5% CHA + | 17.3 |
| 0.5% Farnesol (8) | |

### 6.9. Example 9

Antimicrobial efficacy of farnesol in combination with antimicrobial compounds chlorhexidine free base, chlorhexidine diacetate, and octoxyglycerin on the in vitro airway model.

In Vitro Airway Model

The antimicrobial effect of compositions comprising farnesol in combination with antimicrobial compounds chlorhexidine free base, chlorhexidine diacetate, and octoxyglycerin were evaluated for bacterial colonization on endotracheal (ET) segments in an in vitro airway model as described below.

ET segments (Hi-Lo 7.0 mm) were prepared using the following compositions as disclosed in Example 7.

8. 0.5% CHX+0.5% CHA+0.5% Farnesol dissolved in 69.5% THF and 30% Methanol and containing Polyurethane 93A and Polyurethane 6TD.

9. 0.5% CHX+0.5% CHA+5% Octoxyglycerin dissolved in 65% THF and 30% Methanol and containing Polyurethane 93A and Polyurethane 6TD.

10. 0.5% CHX+0.5% CHA+0.5% Farnesol+5% Octoxyglycerin dissolved in 64.5% THF and 30% Methanol and containing Polyurethane 93A and Polyurethane 6TD.

Table 12 shows that the addition of octoxyglycerin to the impregnating compositions comprising chlorhexidine free base, chlorhexidine diacetate, and farnesol induces a larger zone of inhibition than in the absence of octoxyglycerin in various bacterial strains, including methicillin resistant Staphylococcus aureus (MRSA).

TABLE 12
Zone of inhibition data from coated ET segments

| | Zone of Inhibition (mm) | | | | |
|---|---|---|---|---|---|
| | P. | E. | A. | S. | |
| Group | aeruginosa | aerogenes | baumanni | aureus | MRSA |
| 8 | 17 | 17.3 | 22 | 21.5 | 21.3 |
| 10 | 17.8 | 18.5 | 23.3 | 22.3 | 22.8 |

### 6.10. Example 10

Antimicrobial efficacy of the combination of octoglycerin and farnesol with antimicrobial compounds, chlorhexidine free base, and chlorhexidine salt against various bacterial pathogens in the in vitro airway model.

Results are shown in Tables 13 and 14 below. The combination of octoxyglycerin and farnesol was more effective in combination with chlorhexidine free base and chlorhexidine diacetate than either alone in combination with chlorhexidine free base and chlorhexidine diacetate at preventing bacterial colonization on ET segments. These results are also reflected in the growth of bacteria subcultures from various sections of the segments.

TABLE 13
Bacterial colonization of ET segments on E. aerogenes

| | Bacterial colonization |
|---|---|
| Composition | of ET segments |
| 0.5% CHX + 0.5% CHA + 0.5% | 2.1 × 10³ |
| Farnesol (8) | |
| 0.5% CHX + 0.5% CHA + 5% | 4.2 × 10² |
| Octoxyglycerin (9) | |
| 0.5% CHX + 0.5% CHA + 0.5% | 10 |
| Farnesol + 5% Octoxyglycerin (10) | |
| Control* | ˜10⁶ |

*Uncoated ET without antimicrobial.

TABLE 14
Growth of cultures swabbed from trachea

Subculture from trachea

| Composition | Proximal site | Distal site |
|---|---|---|
| 0.5% CHX + 0.5% CHA + 0.5% | ˜10⁶ | 8 × 10⁵ |
| Farnesol (8) | | |
| 0.5% CHX + 0.5% CHA + 5% | ˜10⁵ | ˜10⁴ |
| Octoxyglycerin (9) | | |
| 0.5% CHX + 0.5% CHA + 0.5% | 1.0 × 10³ | 1.5 × 10² |
| Farnesol + 5% Octoxyglycerin (10) | | |
| Control* | ˜10⁶ | ˜10⁶ |

*Uncoated ET without antimicrobial.

Various publications and have been referenced herein, the contents of which are hereby incorporated by reference in their entireties.

1-41. (canceled)

42. A polymeric medical article treated with a treatment solution comprising between about 1.0 percent and about 20.0 percent of an anti-infective compound, and between about 1.0 percent and about 10.0 percent of octoxyglycerin, wherein the anti-infective compound is selected from a group consisting of chlorhexidine free base, a chlorhexidine salt, triclosan, and an antibiotic, and wherein the treatment solution further comprises salicylic acid.

43. A polymeric medical article treated with a treatment solution comprising between about 1.0 percent and about 20.0 percent of an anti-infective compound, and between about 1.0 percent and about 10.0 percent of octoxyglycerin, wherein the anti-infective compound is selected from a group consisting of chlorhexidine free base, a chlorhexidine salt, triclosan, and an antibiotic, and wherein the treatment solution further comprises a sesquiterpenoid compound.

44. A polymeric medical article treated with a treatment solution comprising between about 0.5 percent and about 5 percent of chlorhexidine, between about 0.05 percent and about 2.0 percent of triclosan, and between about 1.0 percent and about 10.0 percent of octoxyglycerin, and wherein the treatment solution further comprises salicylic acid.

45. A polymeric medical article treated with a treatment solution comprising between about 0.5 percent and about 5 percent of chlorhexidine, between about 0.05 percent and about 2.0 percent of triclosan, and between about 1.0 percent and about 10.0 percent of octoxyglycerin, and wherein the treatment solution further comprises a sesquiterpenoid compound.

46. The polymeric medical article of claim 42 or 44, wherein salicylic acid is present at a concentration of between 0.5 percent and 5 percent.

47. The polymeric medical article of claim 46, wherein salicylic acid is present at a concentration of 1 percent.

48. The polymeric medical article of claim 43 or 45, wherein the sesquiterpenoid compound is present in at a concentration of between 0.1 percent and 2 percent.

49. The polymeric medical article of claim 48, wherein the sesquiterpenoid compound is present at a concentration of 0.5 percent.

50. The polymeric medical article of claim 42 or 44, wherein the medical article is selected from the group consisting of a catheter, the port of a catheter, the hub of a catheter, an arterial graft, a wound dressing, a suture, a wound drainage tube, a soft tissue patch, a glove, a shunt, a stent, a guide wire, a prosthetic device.

51. The polymeric medical article of claim 50, wherein the catheter is selected from the group consisting of a urinary catheter, a vascular catheter, a single lumen central venous catheter, a multiple lumen central venous catheter, a peripherally inserted central venous catheter, an endotracheal catheter, an emergency infusion catheter, a percutaneous sheath introducer system, and a thermodilution catheter.

52. A method of preparing an infection-resistant medical article comprising:
(a) placing the medical article in a treatment solution comprising a solvent selected from the group consisting of water, ethanol, methanol, tetrahydrofuran, and mixtures thereof, wherein the solvent contains between about 0 percent and about 10 percent of a medical polyurethane and about 1.0 percent and about 20 percent of an anti-infective compound and octoxyglycerin for a period of time sufficient to allow the medical article to become coated with the anti-infective compound, wherein the solvent further comprises salicylic acid;
(b) removing the medical article from the treatment solution; and
(c) drying the medical article.

53. A method of preparing an infection-resistant medical article comprising:
(a) placing the medical article in a treatment solution comprising a solvent selected from the group consisting of water, ethanol, methanol, tetrahydrofuran, and mixtures thereof, wherein the solvent contains between about 0 percent and about 10 percent of a medical polyurethane and about 1.0 percent and about 20 percent of an anti-infective compound and octoxyglycerin for a period of time sufficient to allow the medical article to become coated with the anti-infective compound, wherein the solvent further comprises a sesquiterpenoid compound;
(b) removing the medical article from the treatment solution; and
(c) drying the medical article.

54. The method of claim 52 or 53, wherein the anti-infective compound is selected from the group consisting of chlorhexidine free base, a chlorhexidine salt, triclosan, an antibiotic, a silver compound, a trivalent bismuth salt, and mixtures thereof.

55. The method of claim 54, wherein octoxyglycerin is present in the treatment solution at a concentration between about 3 percent and about 7 percent.

56. The method of claim 54, wherein the medical article is dipped in the treatment solution for a period of time sufficient to allow the medical article to incorporate the anti-infective compound.

57. The method of claim 54 further comprising:
(a) placing the dried medical article of element (c) in a coating solution comprising a solvent selected from the group consisting of water, ethanol, methanol, tetrahydrofuran, and mixtures thereof, wherein the solvent contains between about 1.0 percent and about 10 percent of a medical polyurethane, between about 0 percent and about 20 percent of an anti-infective compound, and octoxyglycerin;
(b) removing the medical article from the coating solution; and
(c) drying the medical article.

58. The method of claim 54 further comprising:
(a) placing the dried medical article of element (c) in a coating solution comprising a solvent selected from the group consisting of water, ethanol, methanol, tetrahydrofuran, and mixtures thereof, wherein the solvent contains between about 1.0 percent and about 20 percent of a medical silicone, between about 0 percent and about 20 percent of an anti-infective compound, and otoxyglycerin;
(b) removing the medical article from the coating solution; and
(c) drying the medical article.
